(19) United States
(12) Patent Application Publication
(10) Pub. No.: US 2023/0117142 A1
(43) Pub. Date: Apr. 20, 2023

(54) Instrumented Footwear Device for Upper-Body Exercise Identification

(71) Applicants: Huanfen YAO, Mountain View, CA (US); Alex OLWAL, Stockholm (SE); Abdelkareem BEDRI, Mountain View, CA (US); Google LLC, Mountain View, CA (US)

(72) Inventors: Abdelkareem Bedri, Pittsburgh, PA (US); Alex Olwal, Stockholm (SE); Huanfen Yao, San Carlos, CA (US)

(21) Appl. No.: 17/794,899

(22) PCT Filed: Jan. 24, 2020
(86) PCT No.: PCT/US2020/015020

§ 371 Date: Jul. 22, 2022

(51) Int. Cl. A61B 5/00; A61B 5/11; A61B 5/103

(52) U.S. Cl. CPC A61B 5/6807; A61B 5/1123; A61B 5/1038

(57) ABSTRACT

A computing system configured to perform operations that identify user movement(s) as upper-body exercise(s). The operations can include obtaining sensor data generated by one or more sensors of a footwear device worn by a user, the one or more sensors positioned in one or more positions of the footwear device, the sensor data associated with one or more movements performed by the user. The sensor data can be input into a machine-learned exercise identification model. Exercise identification data can be received as an output of the machine-learned exercise identification model. The exercise identification data can identify the one or more movements performed by the user as one or more upper-body exercises performed by the user, the exercise identification data comprising one or more exercise characteristics associated with each upper-body exercise of the one or more upper-body exercises performed by the user.

### FIELD

The present disclosure relates generally to instrumented footwear devices configured to detect upper-body exercises. More particularly, the present disclosure relates to systems and methods for identifying user movements as one or more upper-body exercises using instrumented footwear device(s) and machine-learned exercise identification model(s).

### BACKGROUND

Exercise enthusiasts commonly desire automatic detection and identification of exercise movements. Identification of upper-body exercises, such as a bench-press or a barbell curl, typically requires a user to wear specific instrumented devices on the upper body of the user. As an example, a user may be required to wear armbands, gloves, or another other upper-body instrumented device to capture data sufficient to identify an upper-body exercise.

However, wearing instrumented upper-body devices has generally been found to frustrate users. Additionally, wearing instrumented upper-body devices can interfere with the performance of certain upper-body exercises. As a result, users typically forego the utilization of instrumented upper-body devices and accordingly lack devices capable of automatically identifying upper-body exercises.

### SUMMARY

Aspects and advantages of embodiments of the present disclosure will be set forth in part in the following description, or can be learned from the description, or can be learned through practice of the embodiments. Aspects of the present disclosure are directed to a method involving the use of technical means such as the computer system or footwear device, and to a device itself (e.g. the computer system, or footwear device). The methods and devices are concerned with performing a technical purpose, that of generating exercise identification data on the basis sensor data obtained from a one or more sensors of footwear devices.

One example aspect of the present disclosure is directed to a computing system including one or more processors and one or more non-transitory computer-readable media that store instructions that when executed by the one or more processors cause the computing system to perform operations. The operations include obtaining sensor data generated by one or more sensors of a footwear device worn by a user, the one or more sensors positioned in one or more positions of the footwear device, the sensor data associated with one or more movements performed by the user. The operations include inputting the sensor data into a machine-learned exercise identification model. The operations include receiving, as an output of the machine-learned exercise identification model, exercise identification data that identifies the one or more movements performed by the user as one or more upper-body exercises performed by the user, the exercise identification data including one or more exercise characteristics associated with each upper-body exercise of the one or more upper-body exercises performed by the user.

Another example aspect of the present disclosure is directed to a footwear device. The footwear device can include one or more sensors, one or more processors, and one or more non-transitory computer-readable media. The one or more non-transitory computer-readable media can store instructions that when executed by the one or more processors cause the footwear device to perform operations. The operations can include obtaining sensor data generated by the one or more sensors. The one or more sensors can be positioned in one or more positions of the footwear device. The sensor data can be associated with one or more movements performed by a user wearing the footwear device. The operations can include inputting the sensor data into a machine-learned exercise identification model. The operations can include receiving, as an output of the machine-learned exercise identification model, exercise identification data that identifies the one or more movements performed by the user as one or more upper-body exercises performed by the user. The exercise identification data can include one or more exercise characteristics associated with each upper-body exercise of the one or more upper-body exercises performed by the user.

Another example aspect of the present disclosure is directed to a computer-implemented method to perform exercise identification. The method can include obtaining, by one or more computing devices, sensor data generated by one or more sensors of a footwear device worn by a user, the one or more sensors positioned in one or more positions of the footwear device, the sensor data associated with one or more movements performed by the user. The method can include inputting, by the one or more computing devices, the sensor data into a machine-learned exercise identification model. The method can include receiving, by the one or more computing devices and as an output of the machine-learned exercise identification model, exercise identification data that identifies the one or more movements performed by the user as one or more upper-body exercises performed by the user, the exercise identification data including one or more exercise characteristics associated with each upper-body exercise of the one or more upper-body exercises performed by the user

Other aspects of the present disclosure are directed to various systems, apparatuses, non-transitory computer-readable media, user interfaces, and electronic devices.

The features disclosed in combination with one aspect of the invention (e.g., the disclosed computing system, etc.) may be combined with other aspects and/or embodiments described herein.

These and other features, aspects, and advantages of various embodiments of the present disclosure will become better understood with reference to the following description and appended claims. The accompanying drawings, which are incorporated in and constitute a part of this specification, illustrate example embodiments of the present disclosure and, together with the description, serve to explain the related principles.

### BRIEF DESCRIPTION OF THE DRAWINGS

FIG. 1A depicts a graphical diagram of an example footwear device according to example embodiments of the present disclosure.

FIGS. 1B-D depict block diagrams of example computing systems that include a footwear device according to example embodiments of the present disclosure.

FIG. 2 depicts a block diagram of an example inference scheme for a machine-learned exercise identification model according to example embodiments of the present disclosure.

FIG. 3 depicts a block diagram of an example training scheme for a machine-learned exercise identification model according to example embodiments of the present disclosure.

FIG. 4 depicts a block diagram of an example computing system for training a machine learned exercise identification model according to example embodiments of the present disclosure.

FIG. 5 is a flowchart depicting an example method of generating user form corrections for user form deficiencies in accordance with example embodiments of the present disclosure.

FIG. 6 is a flowchart depicting an example method for implementing pre-processing and feature extraction of sensor data in accordance with example embodiments of the present disclosure.

### DETAILED DESCRIPTION

### Overview

Example embodiments of the present disclosure are directed to a footwear device and associated computational system that utilizes machine-learned models to interpret sensor data associated with user movement. In particular, the footwear device and associated computational system can identify exercise characteristics associated with performance of one or more exercises by analyzing data collected from sensors embedded in a footwear device that a user has chosen to wear. As an example, the user can perform an upper-body exercise (e.g., a bench-press, etc.) while wearing the footwear device. The footwear device can collect and analyze the sensor data associated with the upper-body exercise to determine exercise characteristics associated with the upper-body exercise (e.g., weight used, repetitions performed, etc.). In such fashion, the footwear device and associated computational system can analyze upper-body focused exercises without the use of motion-tracking devices worn on a user's upper body.

More particularly, the precise identification and analysis of user exercise movements has proven difficult for upper-body focused exercises. Generally, the tracking of such exercise movements necessitates that a user wear one or more upper-body motion tracking devices. As an example, a motion tracking device worn on the wrist and/or shoulder would be required to capture an upper-body focused exercise (e.g., a bicep curl, a shoulder press, etc.). However, wearing such devices can interfere with user exercise performance and generally cause user discomfort. Additionally, motion tracking devices worn on a user's lower body (e.g., a footwear device, ankle device, etc.) have generally been incapable of accurately identifying upper-body exercises. As such, exercise tracking devices configured to track upper-body exercises have previously been disfavored by users and/or have been inaccurate.

In response to this problem, the present disclosure proposes a footwear device that can include a plurality of specialized pressure sensors to collect sensor data associated with a user's movements. This sensor data can be input into a machine-learned exercise identification model to receive exercise identification data that identifies one or more movements performed by the user as one or more upper-body exercises performed by the user. The exercise identification data can include exercise characteristics associated with the upper-body exercises performed by the user. As an example, the exercise identification data may identify a movement performed by the user as a seated bench press exercise. The exercise identification data may further indicate that the user performed ten repetitions of the seated bench press using a weight of 85 pounds. In such fashion, the footwear device and associated computing system can accurately identify specific characteristics associated with any upper-body exercise movement performed by the user without requiring the user to utilize burdensome upper-body motion tracking devices.

More particularly, a computing system can obtain sensor data from a footwear device worn by a user. The footwear device (e.g., a shoe, a boot, an insertable insole, a sock, etc.) can include one or more sensors configured to collect sensor data associated with user movement. The one or more sensors can include pressure sensor(s), accelerometer(s) (e.g., 3-axis accelerometer, etc.), gyroscope(s) (e.g., 3-axis gyroscope, etc.), inertial measurement unit(s) (e.g., a 9-axis inertial measurement unit, a combination of a 3-axis accelerometer and a 3-axis gyroscope, etc.), force-sensitive resistor(s), and/or any other sensor configured to capture sensor data associated with user movement.

In some implementations, the pressure sensor(s) can include a barometer and a covering layer (e.g., rubber, etc.). The barometer can be positioned below and/or inside the covering layer. The covering layer can be made of any material that enables sufficient pressure sensitivity in the barometer (e.g., a rubber layer, a foam layer, a composite layer, etc.). As an example, the footwear device may include a rubber layer (e.g., a rubber sole, a bio-compatible silicone insole, etc.) with one or more pressure sensors covered by the rubber layer. More particularly, the rubber layer can, in some implementations, be cured and embedded with sensor(s) concurrently, allowing the sensor(s) to merge with the structure of the rubber layer. Additionally, the rubber layer can be vacuum pumped to remove air from the structure of the rubber layer. In such fashion, the relatively high density and flexibility of the covering layer (e.g., a rubber layer, foam layer, etc.) enables more accurate transfer of pressure to the one or more barometers. In some implementations, due at least in part to the combination of the barometer and the covering layer, the pressure sensor can be configured to detect pressure less than 0.01 Newtons. In some other implementations, the pressure sensor can be configured to detect pressure at a specificity of 0.1 Newtons.

In some implementations, the one or more sensors can include at least four pressure sensors and at least one inertial measurement unit sensor. The sensors can be placed in discrete area(s) of the footwear device. As an example, four pressure sensors can be placed respectively at an anterior toe area, a posterior heel area, a left-side area (e.g., a metatarsal area, etc.) and a right-side area (e.g., a lateral plantar side area, etc.), while an inertial measurement unit can be placed in a right-side area (e.g., a medial plantar side area). In such fashion, the four pressure sensors and the inertial measurement unit sensor can capture sensor data associated with user movement at a specificity sufficient to accurately identify upper-body exercises. It should be noted, however, that in some alternative implementations the pressure sensors can be placed in any additional and/or alternative area(s). As an example, the footwear device can include 16 pressure sensors, each of the 16 sensors respectively placed in different areas of the footwear device.

The sensor data collected by the sensor(s) can be associated with user movement. The sensor data can describe user weight distribution, force applied by the user, user acceleration, and any other metric relevant to analyzing a user's movements. As an example, the sensor data can describe how much weight the user is placing at certain area(s) of the footwear device. As another example, the sensor data can describe how much force the user is applying at certain times while performing an upper-body exercise. As yet another example, the sensor data can describe an acceleration associated with the user moving a foot. In such fashion, the sensor data can be used to determine if an upper-body exercise is being performed by the user, and if so, what upper-body exercise(s) are being performed by the user.

In some implementations, a filter can be applied to the data channels of one or more sensors. As an example, a filter (e.g., band-pass filter, high-pass filter, low-pass filter, etc.) can be applied to the data channel of each pressure sensor. As another example, a filter can be applied to a data channel of a gyroscope and accelerometer. In some implementations, the filter can be configured to remove drift signal and/or high frequency noise from the data channels. As an example, the filter can be configured to remove drift signal below 0.2 Hz and remove high-frequency noise above 3 Hz. It should be noted that any sort of filter can be applied to the data channel(s) of sensor(s) for attenuation and/or amplification.

In some implementations, one or more features can be extracted from the data channels of one or more sensors. The one or more features can include statistical feature(s) (e.g., mean, median, absolute median, variance, root mean square, entropy, etc.), frequency feature(s) (e.g., power spectrum density, dominant frequency, dominant frequency energy, DC offset, etc.), and/or auto-correlation feature(s) (e.g., maximum autocorrelation peak, number of autocorrelation peaks, height of a first autocorrelation peak after a first zero crossing, etc.). As an example, 14 different features can be extracted from each data channel of each sensor of a footwear device.

The sensor data associated with one or more movements performed by the user can be input into a machine-learned exercise identification model. In some implementations, the one or more feature(s) extracted from the data channel(s) can be input into the machine-learned exercise identification model. The machine-learned exercise identification model can be or can otherwise include one or more neural networks (e.g., deep neural networks) or the like. Neural networks (e.g., deep neural networks) can be feed-forward neural networks, convolutional neural networks, and/or various other types of neural networks. Additionally, or alternatively, the machine-learned exercise model can be or can otherwise include one or more classifier models (e.g., random forest classifier, etc.).

Exercise identification data can be received as an output of the machine-learned exercise identification model. The exercise identification data can identify the one or more movements performed by the user as one or more upper-body exercises performed by the user. As an example, the exercise identification data can identify one or more movements as a seated dumbbell shoulder press exercise performed by the user. As another example, the exercise identification data can identify one or more movements as a seated dumbbell shoulder press exercise and a bicep curl exercise performed by the user (e.g., performed sequentially). As yet another example, the exercise identification data can identify one or more movements as a barbell clean exercise and a barbell shoulder press exercise performed by the user (e.g., performed as part of one exercise circuit). In such fashion, the exercise identification data can identify any combination of upper-body exercise performed by the user over a period of time.

An upper-body exercise can be defined as an exercise that primarily requires utilization of upper-body muscles located above the waist of a user to perform. As an example, performing a bench-press exercise can be categorized as an upper-body exercise because the bench-press primarily requires utilization of upper-body muscles located above the waist of the user (deltoids, triceps, pectorals, etc.). Although the bench-press exercise can also require utilization of lower-body muscles (e.g., quadriceps, etc.), the primary muscles utilized during performance of the exercise are upper-body muscles. As another example, performing a pullup exercise can be categorized as an upper-body exercise because the pullup primarily requires utilization of upper-body muscles located above the user's waist (e.g., biceps, trapezius, latissimus dorsi, etc.). It should be noted that although weightlifting exercises are discussed primarily to illustrate the functionality of the embodiments, any other sort of upper-body exercise (e.g., calisthenics, stretches, etc.) can be identified by the exercise identification data.

The exercise identification data can include one or more exercise characteristics associated with each upper-body exercise of the one or more upper-body exercises performed by the user. In some implementations, the exercise characteristics include an amount of time spent on each upper-body exercise by the user. As an example, the exercise characteristics can indicate that a user spent 35 seconds performing a bench-press exercise. As another example, the exercise characteristics can indicate that a user performed a 10-repetition shoulder-press exercise in 4.8 seconds. As yet another example, the exercise characteristics can indicate that the user spent 3 minutes performing an upper-body exercise circuit including five different upper-body exercises. In some implementations, the amount of time can include the amount of time resting between upper-body exercises. As an example, the exercise characteristics can indicate the user spent 50 seconds resting between individual sets of an upper-body exercise.

In some implementations, the exercise characteristics can include a number of repetitions performed for each upper-body exercise. As an example, the exercise characteristics can indicate that the user performed 12 repetitions for a lateral raise exercise. As another example, the exercise characteristics can indicate that the user performed 5 repetitions for a clean and jerk full-body exercise and 3 repetitions for a press exercise. In some implementations, the number of repetitions performed can exclude repetitions that are not fully completed by the user. As an example, the number of repetitions can exclude a repetition that does not reach the highest point of an upper-body weightlifting exercise (e.g., a highest point (e.g., a lockout point) of a bench press exercise, etc.). The exercise characteristics can indicate a number of failed repetitions associated with upper-body exercises performed by the user.

In some implementations, the exercise characteristics can include an exercise categorization that further categorizes an upper-body exercise as a full-body exercise. A full-body exercise can be defined as an upper-body exercise that additionally requires significant utilization of lower-body muscles (e.g., muscles located below the waist of a user) to perform. As an example, performing a clean and jerk exercise can require significant utilization of lower-body muscles (e.g., gluteus maximus, etc.). In such fashion, the exercise identification data is not limited to identifying only upper-body exercises that require minimal lower-body muscle utilization.

It should be noted that, in addition to upper-body and full-body exercises, the present embodiments can, in some implementations additionally detect any form of lower-body exercise. As an example, one or more movements can be identified as lower-body exercise(s) by the exercise identification model, and can be categorized accordingly. For example, the exercise identification model may identify one or more user movements as a squat exercise. For another example, the exercise identification model can identify one or more user movements as a deadlift exercise. Exercise characteristics can categorize the deadlift exercise according to the user's performance of the exercise (e.g., as an upper-body exercise, full-body exercise, lower-body exercise, etc.).

In some implementations, the one or more exercise characteristics can include a weight used for each upper-body exercise performed by the user. As an example, the exercise characteristics can indicate that the user used 85 pounds for performing a bench press exercise. As another example, the exercise characteristics can indicate that the user used 300 pounds for performing a press exercise and 365 pounds for performing a clean exercise. As yet another example, the exercise characteristics can indicate that the user used 65 pounds, 45 pounds, and 25 pounds for performing one set of a shoulder press exercise circuit. In such fashion, the exercise characteristics can associate a weight used for each repetition performed by the user.

In some implementations, one or more exercise form deficiencies associated with the user's performance of upper-body exercises can be determined. The exercise form deficiencies can be determined based on the sensor data and the exercise identification data (e.g., weight distribution of the user, force applied by the user, user limb positioning, amount of time, etc.). The exercise form deficiencies can include a difference between the user's performance of one or more upper-body exercises and an optimal performance of the one or more upper-body exercises. As an example, an optimal form for a clean exercise can include a knee positioning that does not extend over the vertical plane of the user's toe. If the user extends his knee past the vertical plane of the toe during the performance of a clean exercise, an exercise form deficiency can be determined based on a difference between the optimal knee positioning and the users knee positioning. As another example, an optimal form for a bench press can include an optimal amount of force applied to the ground. If the user fails to apply this amount of force to the ground during the performance of a bench press exercise (e.g., the user's feet are not located firmly on the ground), an exercise form deficiency can be determined based on a difference between the optimal amount of force applied to the ground and the user's amount of force applied to the ground.

In some implementations, one or more user form corrections can be generated based on the one or more exercise form deficiencies. As an example, for an exercise form deficiency associated with the user extending their knee over the vertical plane of the toe while performing a clean exercise, a user form correction can be generated that advises the user to place more weight on the heel of their foot while performing a clean exercise. As another example, for an exercise form deficiency associated with the user not applying enough force to the ground while performing a bench press exercise, a user form correction can be generated that advises the user to apply more force to the ground with their feet while performing a bench press exercise. In some implementations, the one or more form corrections can be communicated to the user and/or provided for display to the user (e.g., through an application associated with the footwear device, through a user display device, etc.).

In some implementations, the machine-learned exercise identification model can be a user-independent generalized model. More particularly, the machine-learned exercise identification model can be trained based on a generalized, user-agnostic set of training data. In such fashion, the machine-learned exercise identification model does not necessarily need to be trained based on movements and/or movement patterns specific to a user, and therefore can identify exercises for any user who utilizes the associated footwear device.

In some implementations, a loss function that evaluates a difference between the exercise identification data and actual exercise data can be evaluated. The actual exercise data can be provided by a user. As an example, the exercise identification data can identify the one or more movements performed by the user as a bench press exercise. The user can provide actual exercise data that identifies the one or more movements performed by the user as a seated pullover exercise. The loss function can evaluate the difference between the exercise identification data. The loss function can be backpropagated through the machine-learned exercise identification model to modify values of one or more parameters of the machine-learned exercise identification model (e.g., based on a gradient of the loss function).

The present disclosure provides a number of technical effects and benefits. As one example, the proposed footwear device allows for accurate tracking and identification of upper-body exercises without necessitating the use of additional motion tracking devices. As another example, the proposed footwear device can enable automatic tracking of detailed exercise identification data (e.g., exercises performed, weight used, form corrections, etc.). The automatic collection of advanced exercise tracking data can significantly reduce user-device interactions required to manually enter exercise tracking information into a user device. The detection of exercise form inefficiencies can prevent serious harm to users from performing exercises with poor form.

Another aspect of the present disclosure is directed to a machine-learned exercise identification model. The machine-learned exercise identification model can be configured to receive data generated by one or more sensors of a footwear device worn by a user, the one or more sensors positioned in one or more positions of the footwear device, the sensor data associated with one or more movements performed by the user. The machine-learned exercise identification model can be further configured to generate, as an output of the machine-learned exercise identification model, exercise identification data that identifies the one or more movements performed by the user as one or more upper-body exercises performed by the user, the exercise identification data including one or more exercise characteristics associated with each upper-body exercise of the one or more upper-body exercises performed by the user.

Another aspect of the present disclosure is directed to a method of training a machine-learned exercise identification model. The trained machine-learned exercise identification model can be configured to receive data generated by one or more sensors of a footwear device worn by a user, the one or more sensors positioned in one or more positions of the footwear device, the sensor data associated with one or more movements performed by the user. The trained machine-learned exercise identification model can be further configured to generate, as an output of the machine-learned exercise identification model, exercise identification data that identifies the one or more movements performed by the user as one or more upper-body exercises performed by the user, the exercise identification data including one or more exercise characteristics associated with each upper-body exercise of the one or more upper-body exercises performed by the user. The method of training the machine-learned exercise identification model can include receiving training data and actual exercise data. The method of training the machine-learned exercise identification model can include generating test exercise identification data including one or more test exercise characteristics associated with the training data. The method of training the machine-learned exercise identification model can include evaluating a loss function that evaluates a difference between the test exercise identification data and the actual exercise data. The method of training the machine-learned exercise identification model can include modifying values for one or more parameters of the machine-learned exercise identification model based on the loss function

With reference now to the Figures, example embodiments of the present disclosure will be discussed in further detail.

### Example Devices and Systems

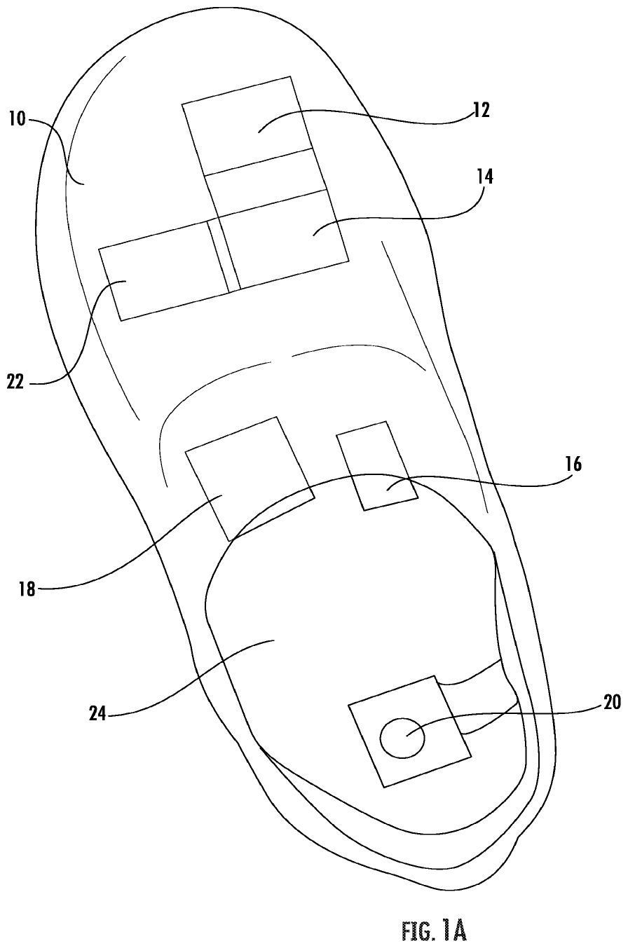

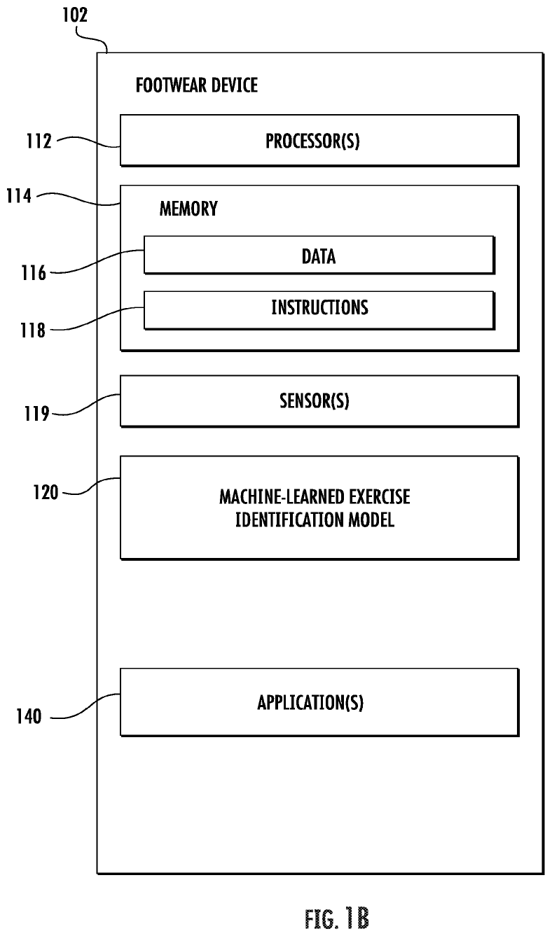

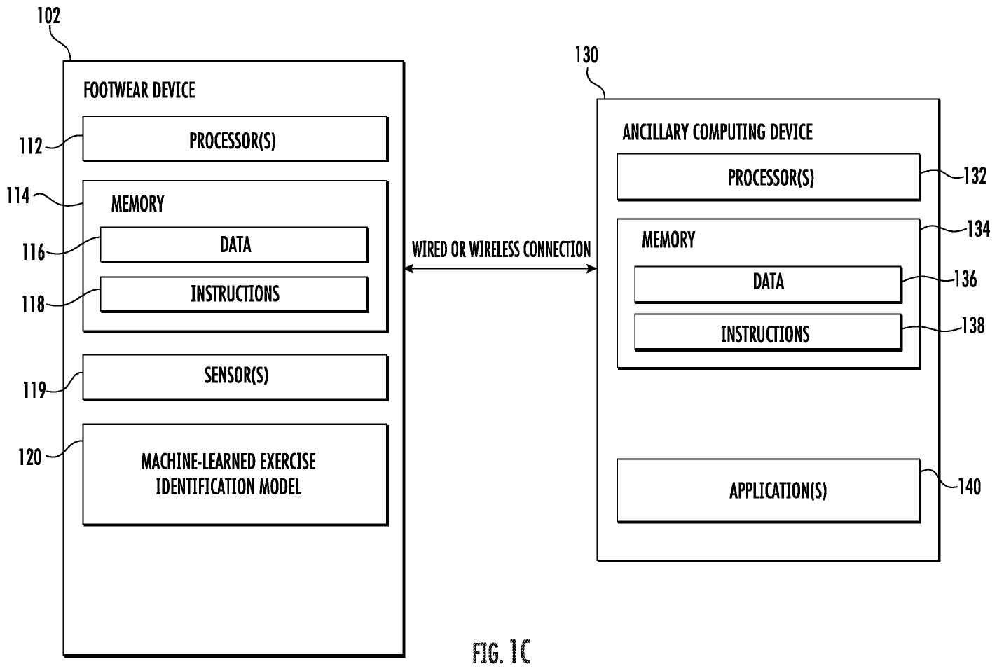

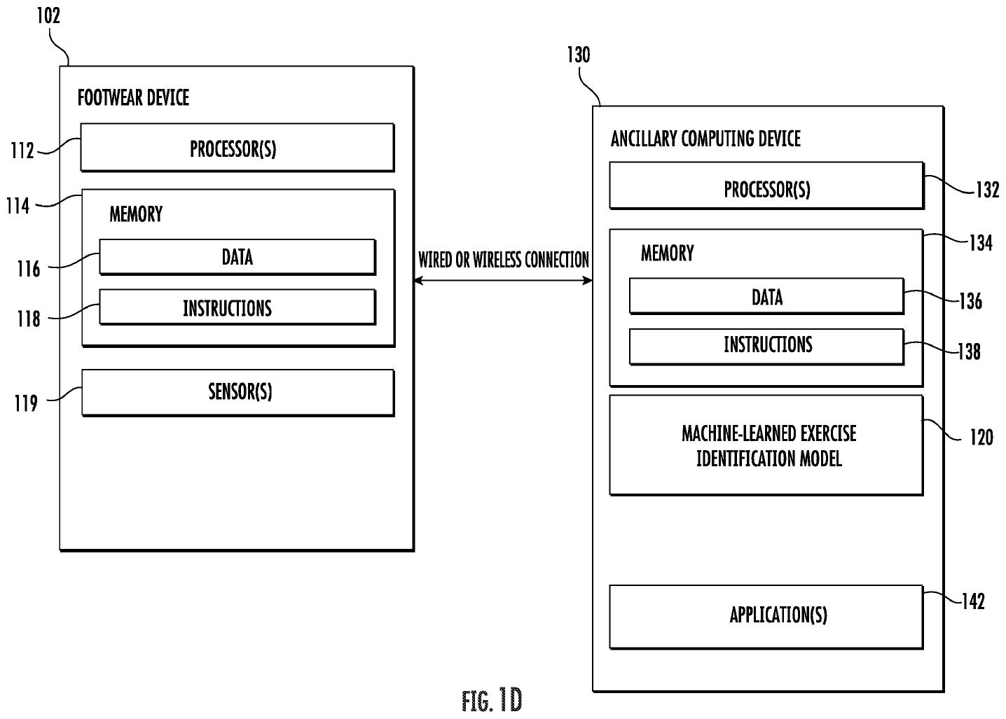

FIG. 1A depicts a graphical diagram of an example footwear device 10 according to example embodiments of the present disclosure. Although the footwear device 10 is depicted as a shoe, it should be noted that the footwear device 10 can utilize any other form (e.g., a boot, an insertable sole and/or insole, a sock, an external shoe covering, etc.). The footwear device 10 can include one or more sensors (e.g., sensors and/or sensor clusters 12-22) that generate sensor data that describes one or more movements performed by a user. The one or more sensors can include pressure sensor(s), accelerometer(s) (e.g., 3-axis accelerometer, etc.), gyroscope(s) (e.g., 3-axis gyroscope, etc.), inertial measurement unit(s) (e.g., a 9-axis inertial measurement unit), force-sensitive resistor(s), and/or any other sensor configured to capture sensor data associated with user movement. In some implementations, the pressure sensor(s) can include a barometer and a rubber layer (e.g., rubber layer 24). The barometer can be positioned below and/or inside the rubber layer. As an example, the footwear device may include a rubber layer (e.g., a rubber sole, a bio-compatible silicone insole, etc.) with one or more pressure sensors covered by the rubber layer.

The sensor data collected by the sensor(s) can be associated with user movement. The sensor data can describe user weight distribution, force applied by the user, user acceleration, and any other metric relevant to analyzing a user's movements. As an example, the sensor data can describe how much weight the user is placing at certain points. As another example, the sensor data can describe how much force the user is applying at certain times while performing an upper-body exercise. As yet another example, the sensor data can describe an acceleration associated with the user moving a foot. In such fashion, the sensor data can be used to determine if an upper-body exercise is being performed by the user, and if so, what upper-body exercise(s) are being performed by the user. As will be discussed in greater detail with reference to FIG. 1B, the sensor data can, in some implementations, undergo pre-processing and feature extraction.

As one specific example, the footwear device 10 can include one or more sensors positioned in different areas of footwear device. As an example, a pressure sensor 12 can be placed at an anterior toe area, a pressure sensor 14 can be placed at a right-side area (e.g., a medial plantar area), a pressure sensor 20 can be placed at a posterior heel area (e.g., a tibial area), and an inertial measurement unit 16 can be placed at a right-side area (e.g., a medial arch area). It should be noted that the above pressure sensors can also be, additionally or alternatively, any other combination of sensors configured to measure user movement. As an example, the sensor(s) 18 placed at a left-side area (e.g., a lateral plantar area) can be a cluster of three sensors (e.g., a pressure sensor, an IMU sensor, a force-sensitive resistor, etc.). As another example, the sensor(s) 22 placed at a left-side area (e.g., a lateral plantar area) can be a second inertial measurement unit.

Although a certain number of sensors are depicted in the above example, any number and/or type of sensor(s) can be utilized to measure user movement at a specificity sufficient to identify upper-body exercises. As an example, the footwear device 10 can include 16 different sensor clusters, each sensor cluster including a pressure sensor and an IMU and placed in an area of the footwear device (e.g., a right-side area, a left-side area, an anterior toe area, a posterior heel area, etc.).

The sensors of the footwear device (e.g., sensors 12-22) can be located below and/or inside a rubber layer 24. The rubber layer 24 can be a layer of the footwear device that contacts the foot of the user (e.g., a rubber sole, a bio-compatible silicone insole, etc.) and/or is placed in close proximity to the foot of the user (e.g., a layer below the sole of the footwear device). More particularly, the rubber layer 24 can, in some implementations, be cured and embedded with sensor(s) concurrently, allowing the sensor(s) to merge with the structure of the rubber layer. Additionally, the rubber layer 24 can be vacuum pumped to remove air from the structure of the rubber layer. In such fashion, the relatively high density and flexibility of the rubber layer enables more accurate transfer of pressure to the one or more pressure sensors and/or other sensor(s). In some implementations, this can enable the pressure sensor(s) to detect pressure less than 0.01 Newtons, therefore allowing for more accurate identification of upper-body exercises by the footwear device 10.

FIGS. 1B-D depict block diagrams of example computing systems that include a footwear device according to example embodiments of the present disclosure. Referring specifically to FIG. 1B, an example footwear device 102 can include one or more processors 112 and a memory 114. The one or more processors 112 can be any suitable processing device (e.g., a processor core, a microprocessor, an ASIC, a FPGA, a controller, a microcontroller, etc.) and can be one processor or a plurality of processors that are operatively connected. The memory 114 can include one or more non-transitory computer-readable storage mediums, such as RAM, ROM, EEPROM, EPROM, flash memory devices, magnetic disks, etc., and combinations thereof. The memory 114 can store data 116 and instructions 118 which are executed by the processor 112 to cause the footwear device 102 to perform operations.

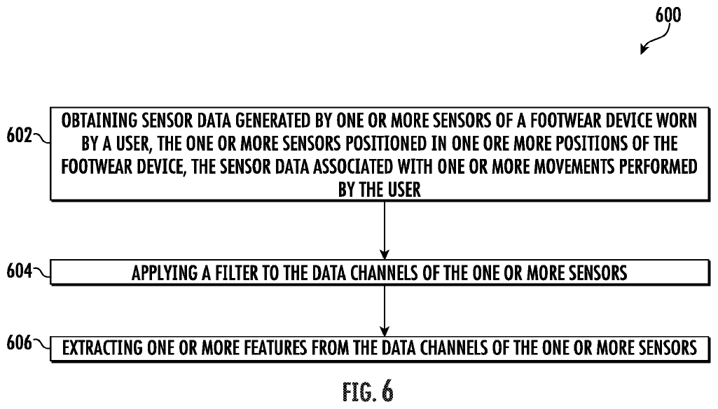

The footwear device 102 can include one or more sensors 119. At least some of the sensors 119 can generate sensor data associated with one or more movements performed by a user, as described previously with reference to FIG. 1A. In some implementations, the sensor data from the sensor(s) can undergo pre-processing (e.g., data filtering, etc.) and feature extraction, as will be discussed in greater detail with reference to FIG. 6.

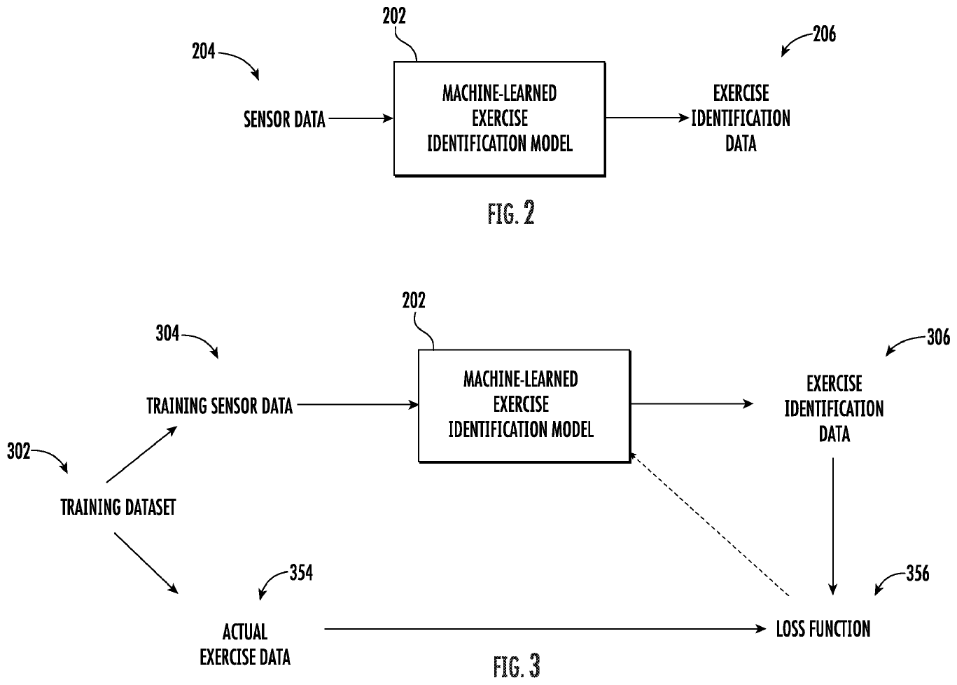

In some implementations, the footwear device 102 can store and implement one or more machine-learned models 120. For example, the machine-learned models 120 can be or can otherwise include various machine-learned models such as neural networks (e.g., deep neural networks) or other types of machine-learned models, including non-linear models and/or linear models. Neural networks can include feed-forward neural networks, recurrent neural networks (e.g., long short-term memory recurrent neural networks), convolutional neural networks or other forms of neural networks. Example machine-learned models 120 are discussed with reference to FIGS. 2-3.

In some implementations, the footwear device 102 can store and implement one or more applications 140. The applications 140 can be any applications including, as examples, an exercise tracking application, a social media application, a health and wellness application, etc. In some implementations, the applications 140 can receive exercise identification data from the machine-learned exercise identification model 120 and perform various operations (e.g., via one or more application programming interfaces). As an example, a social media application may, if a user chooses to enable such functionality, automatically post a message to the user's social media feed describing the upper-body exercises identified by the exercise identification data. As another example, an exercise tracking application may record the sets and repetitions performed for each upper-body exercise described by the exercise identification data.

Further to the descriptions herein, a user may be provided with controls allowing the user to make an election as to both if and when systems, programs, or features described herein may enable collection of user information (e.g., information about a user's social network, social actions, or activities, a user's preferences, or a user's current location), and if the user is sent content or communications from a server. In addition, certain data may be treated in one or more ways before it is stored or used, so that personally identifiable information is removed. For example, a user's identity may be treated so that no personally identifiable information can be determined for the user, or a user's geographic location may be generalized where location information is obtained (such as to a city, ZIP code, or state level), so that a particular location of a user cannot be determined. Thus, the user may have control over what information is collected about the user, how that information is used, and what information is provided to the user.

Referring now to FIGS. 1C and 1D, in some implementations, a portion of the components described with reference to FIG. 1B can be located in an ancillary computing device 130 rather than the footwear device 102. For example, this can be done to make the footwear device 102 lighter, cheaper, smaller, etc.

As one example, with reference to FIG. 1C, the model 120 remains at the footwear device 102 but the application(s) 140 are stored at and implemented by the ancillary computing device 130. In this scenario, the footwear device 102 can transmit the interpretation provided by the model 120 to the ancillary computing device 130.

As another example, with reference to FIG. 1D, all of the model 120 and the application(s) 140 are stored at and implemented by the ancillary computing device 130. In this scenario, the footwear device 102 can transmit the sensor data generated by the sensor(s) 119 to the ancillary computing device 130. As yet another example, with reference to FIG. 1C and 1D, pre-processing (e.g., data filtering, etc.) and feature extraction of sensor data can be implemented by the ancillary computing device 130, as will be discussed in greater detail with reference to FIG. 6.

The ancillary computing device 130 can be any different type of computing device including smartphone, tablet, laptop, server computing device, computing device that can be worn, embedded computing device, vehicle computing device, navigational computing device, gaming console, etc.

In some implementations, the footwear device 102 and/or ancillary computing device 130 can include network interface(s) to connect to the larger Internet as a whole (e.g., via a data and/or Wi-Fi connection).

### Example Model Arrangements

FIG. 2 depicts a block diagram of an example inference scheme for a machine-learned exercise identification model 202 according to example embodiments of the present disclosure. The machine-learned exercise identification model 202 can receive and process sensor data 204 that is associated with a user's movement. The machine-learned exercise identification model 202 can process the sensor data 204 to generate exercise identification data 206.

As examples, the exercise identification data 206 can identify the one or more movements associated with sensor data 204 as one or more upper-body exercises performed by the user. As an example, the exercise identification data 206 can identify one or more movements as a seated dumbbell shoulder press exercise performed by the user. As another example, the exercise identification data 206 can identify one or more movements as a seated dumbbell shoulder press exercise and a bicep curl exercise performed by the user (e.g., performed sequentially). As yet another example, the exercise identification data 206 can identify one or more movements as a barbell clean exercise and a barbell shoulder press exercise performed by the user (e.g., performed as part of one exercise circuit). In such fashion, the exercise identification data 206 can identify any combination of upper-body exercise performed by the user over a period of time.

An upper-body exercise can be defined as an exercise that primarily requires utilization of upper-body muscles located above the waist of a user to perform. As an example, performing a bench-press exercise can be categorized as an upper-body exercise because the bench-press primarily requires utilization of upper-body muscles located above the waist of the user (deltoids, triceps, pectorals, etc.). Although the bench-press exercise can also require utilization of lower-body muscles (e.g., quadriceps, etc.), the primary muscles utilized during performance of the exercise are upper-body muscles. As another example, performing a pullup exercise can be categorized as an upper-body exercise because the pullup primarily requires utilization of upper-body muscles located above the user's waist (e.g., biceps, trapezius, latissimus dorsi, etc.). It should be noted that although weightlifting exercises are discussed primarily to illustrate the functionality of the embodiments, any other sort of exercise (e.g., calisthenics, stretches, etc.) can be identified by the exercise identification data.

In some implementations, the exercise identification data 206 can further categorize an upper-body exercise as a full-body exercise. A full-body exercise can be defined as an upper-body exercise that additionally requires significant utilization of lower-body muscles (e.g., muscles located below the waist of a user) to perform. As an example, performing a clean and jerk exercise can require significant utilization of lower-body muscles (e.g., gluteus maximus, etc.). In such fashion, the exercise identification data is not limited to identifying only upper-body exercises that require minimal lower-body muscle utilization.

The exercise identification data 206 can include one or more exercise characteristics associated with each upper-body exercise of the one or more upper-body exercises performed by the user. In some implementations, the exercise characteristics include an amount of time spent on each upper-body exercise by the user. As an example, the exercise characteristics can indicate that a user spent 35 seconds performing a bench-press exercise. As another example, the exercise characteristics can indicate that a user performed a 10-repetition shoulder-press exercise in 4.8 seconds. As yet another example, the exercise characteristics can indicate that the user spent 3 minutes performing an upper-body exercise circuit including five different upper-body exercises. In some implementations, the amount of time can include the amount of time resting between upper-body exercises. As an example, the exercise characteristics can indicate the user spent 50 seconds resting between individual sets of an upper-body exercise.

In some implementations, the exercise characteristics can include a number of repetitions performed for each upper-body exercise. As an example, the exercise characteristics can indicate that the user performed 12 repetitions for a lateral raise exercise. As another example, the exercise characteristics can indicate that the user performed 5 repetitions for a clean and jerk full-body exercise and 3 repetitions for a press exercise. In some implementations, the number of repetitions performed can exclude repetitions that are not fully completed by the user. As an example, the number of repetitions can exclude a repetition that does not reach the highest point of an upper-body weightlifting exercise (e.g., a highest point (e.g., a lockout point) of a bench press exercise, etc.). The exercise characteristics can indicate a number of failed repetitions associated with upper-body exercises performed by the user.

In some implementations, the one or more exercise characteristics can include a weight used for each upper-body exercise performed by the user. As an example, the exercise characteristics can indicate that the user used 85 pounds for performing a bench press exercise. As another example, the exercise characteristics can indicate that the user used 300 pounds for performing a press exercise and 365 pounds for performing a clean exercise. As yet another example, the exercise characteristics can indicate that the user used 65 pounds, 45 pounds, and 25 pounds for performing one set of a shoulder press exercise circuit. In such fashion, the exercise characteristics can associate a weight used for each repetition performed by the user.

Referring now to FIG. 3, in some implementations, the machine-learned exercise identification model 202 can be trained using an supervised learning approach. A training dataset 302 can include sets of training sensor data 304 (e.g., sensor data captured by sensors 119) that was collected while a person was performing a known upper-body exercise with a ground truth interpretation. Therefore, the exercise identification data 306 provided by the model 202 on each set of training sensor data 304 can be compared to the ground truth interpretation (e.g., actual exercise data) 354 using a loss function 356. The model 202 can be trained using the loss function 356 (e.g., a gradient thereof). For example, the values of the parameters of the model 202 can be updated as the loss function 356 is backpropagated through the model 202.

In some implementations, the machine-learned exercise identification model 202 can be trained, additionally or alternatively, using an unsupervised learning approach. As an example, the footwear device can collect sensor data associated with a series of motions that indicate exercise performance (e.g., rapid motion, repetitive motion, etc.). A user of the footwear device can label the exercise that was performed. The user label can be input to the machine-learned exercise identification model 202 alongside the collected sensor data to train the machine-learned exercise identification model in an unsupervised fashion. Thus, the machine-learned exercise identification model can be trained using an unsupervised training technique to reinforce exercise identification training and/or provide a base level of exercise identification functionality.

In some implementations, the training dataset 302 can include data associated with a user training/on-boarding procedure in which a new user is asked to perform certain upper-body exercises (e.g., e.g., a bench-press, a shoulder-press, a bicep curl, etc.). If the user chooses to do so, the model 202 can be trained, re-trained, refined, personalized, etc. based on the actual exercise data provided by the user (e.g., using the supervised scheme shown in FIG. 3). As an example, exercise data can be collected in 10 3-second windows for 30 seconds of user exercise performance of a single exercise. 2 of these 10 windows can be selected for testing while 8 can be used for training the model. This can enable the exercise identification model 202 to have improved accuracy for a specific user.

Additionally or alternatively, in some implementations, the exercise identification model 202 can first be trained on generic upper-body exercise examples. The model trained on normal exercises can then be refined via transfer learning (fine-tuning) to train on the actual exercise data provided by the user. In some implementations, instead of modifying higher layers, when switching to actual exercise data, lower layers can simply be added to the bottom of the transferred model. These new layers can be trained on the smaller dataset collected via the sensors of the footwear device from a variety of users. Using this data, the machine-learned exercise identification model 202 can be trained in a generalized manner that allows for identification of upper-body exercises for any user. In such fashion, a new user of the footwear device 102 is not required to spend additional time to initially train the machine-learned exercise identification model 202.

### Example Training Environment

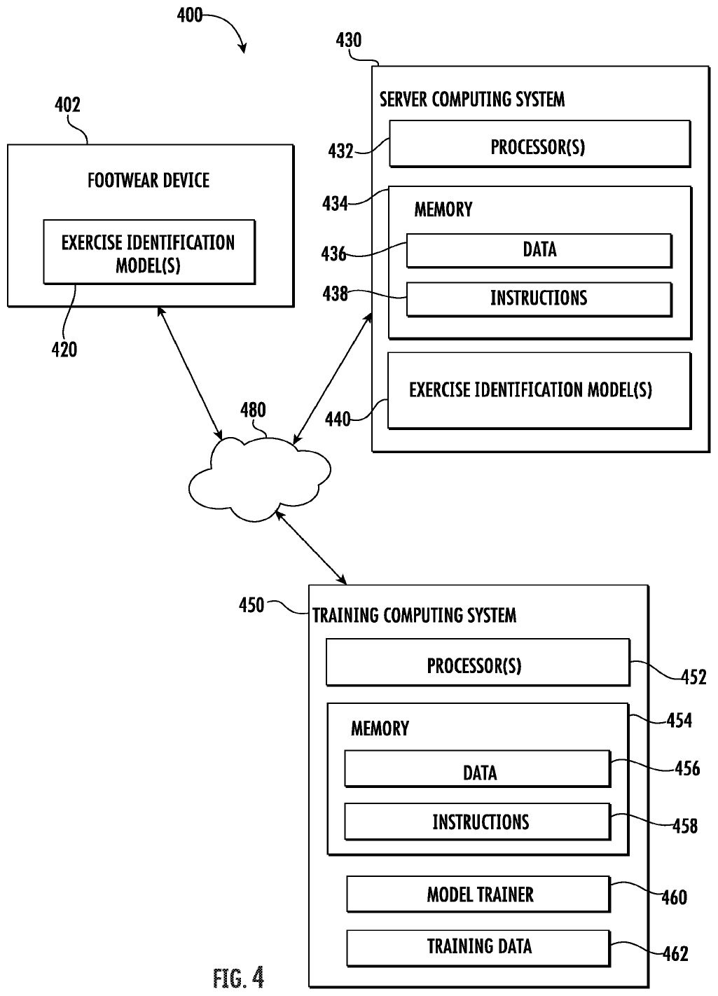

FIG. 4 depicts a block diagram of an example computing system 400 for training machine-learned models according to example embodiments of the present disclosure. The system 400 includes a footwear device 402, a server computing system 430, and a training computing system 450 that are communicatively coupled over a network 480.

The footwear device 402 can be any type of footwear device suitable to contain one or more sensors (e.g., a boot, shoe, sandal, external shoe covering, footwear addition, etc.). The footwear device 402 can include one or more sensors and, in some implementations, can include one or more computing devices sufficient to enable the utilization of machine-learned exercise identification model(s) 420.

In some implementations, the footwear device 402 can store or include one or more machine-learned models 420. For example, the machine-learned models 420 can be or can otherwise include various machine-learned models such as neural networks (e.g., deep neural networks) or other types of machine-learned models, including non-linear models and/or linear models. Neural networks can include feed-forward neural networks, recurrent neural networks (e.g., long short-term memory recurrent neural networks), convolutional neural networks or other forms of neural networks.

In some implementations, the one or more machine-learned models 420 can be received from the server computing system 430 over network 480 and then stored and implemented at the footwear device 402. In some implementations, the footwear device 402 can implement multiple parallel instances of a single machine-learned model 420 (e.g., to perform parallel exercise identifications across multiple instances of sensor data).

Additionally or alternatively, one or more machine-learned models 440 can be included in or otherwise stored and implemented by the server computing system 430 that communicates with the footwear device 402 according to a client-server relationship. For example, the machine-learned models 440 can be implemented by the server computing system 440 as a portion of a web service (e.g., an exercise identification service). Thus, one or more models 420 can be stored and implemented at the footwear device 402 and/or one or more models 440 can be stored and implemented at the server computing system 430.

The footwear device 402 can also include one or more user input component 422 that receives user input. For example, the user input component 422 can be a touch-sensitive component (e.g., a touch pad, a motion sensor, etc) that is sensitive to the touch of a user input object (e.g., a finger or a stylus). Other example user input components include a microphone, a button, or other means by which a user can provide user input.

The server computing system 430 includes one or more processors 432 and a memory 434. The one or more processors 432 can be any suitable processing device (e.g., a processor core, a microprocessor, an ASIC, a FPGA, a controller, a microcontroller, etc.) and can be one processor or a plurality of processors that are operatively connected. The memory 434 can include one or more non-transitory computer-readable storage mediums, such as RAM, ROM, EEPROM, EPROM, flash memory devices, magnetic disks, etc., and combinations thereof. The memory 434 can store data 436 and instructions 438 which are executed by the processor 432 to cause the server computing system 430 to perform operations.

In some implementations, the server computing system 430 includes or is otherwise implemented by one or more server computing devices. In instances in which the server computing system 430 includes plural server computing devices, such server computing devices can operate according to sequential computing architectures, parallel computing architectures, or some combination thereof.

As described above, the server computing system 430 can store or otherwise include one or more machine-learned models 440. For example, the models 440 can be or can otherwise include various machine-learned models. Example machine-learned models include neural networks or other multi-layer non-linear models. Example neural networks include feed forward neural networks, deep neural networks, recurrent neural networks, and convolutional neural networks.

The footwear device 402 and/or the server computing system 430 can train the models 420 and/or 440 via interaction with the training computing system 450 that is communicatively coupled over the network 480. The training computing system 450 can be separate from the server computing system 430 or can be a portion of the server computing system 430.

The training computing system 450 includes one or more processors 452 and a memory 454. The one or more processors 452 can be any suitable processing device (e.g., a processor core, a microprocessor, an ASIC, a FPGA, a controller, a microcontroller, etc.) and can be one processor or a plurality of processors that are operatively connected. The memory 454 can include one or more non-transitory computer-readable storage mediums, such as RAM, ROM, EEPROM, EPROM, flash memory devices, magnetic disks, etc., and combinations thereof. The memory 454 can store data 456 and instructions 458 which are executed by the processor 452 to cause the training computing system 450 to perform operations. In some implementations, the training computing system 450 includes or is otherwise implemented by one or more server computing devices.

The training computing system 450 can include a model trainer 460 that trains the machine-learned models 420 and/or 440 stored at the footwear device 402 and/or the server computing system 430 using various training or learning techniques, such as, for example, backwards propagation of errors. For example, a loss function can be backpropagated through the model(s) to update one or more parameters of the model(s) (e.g., based on a gradient of the loss function). Various loss functions can be used such as mean squared error, likelihood loss, cross entropy loss, hinge loss, and/or various other loss functions. Gradient descent techniques can be used to iteratively update the parameters over a number of training iterations.

In some implementations, performing backwards propagation of errors can include performing truncated backpropagation through time. The model trainer 460 can perform a number of generalization techniques (e.g., weight decays, dropouts, etc.) to improve the generalization capability of the models being trained.

In particular, the model trainer 460 can train the machine-learned models 420 and/or 440 based on a set of training data 462. The training data 462 can include, for example, supervised training data, including user-specific supervised training data generated, for example, during a calibration routine.

Thus, in some implementations, if the user has provided consent, the training examples can be provided by the footwear device 402. Thus, in such implementations, the model 420 provided to the footwear device 402 can be trained by the training computing system 450 on user-specific data received from the footwear device 402. In some instances, this process can be referred to as personalizing the model.

The model trainer 460 includes computer logic utilized to provide desired functionality. The model trainer 460 can be implemented in hardware, firmware, and/or software controlling a general purpose processor. For example, in some implementations, the model trainer 460 includes program files stored on a storage device, loaded into a memory and executed by one or more processors. In other implementations, the model trainer 460 includes one or more sets of computer-executable instructions that are stored in a tangible computer-readable storage medium such as RAM hard disk or optical or magnetic media.

The network 480 can be any type of communications network, such as a local area network (e.g., intranet), wide area network (e.g., Internet), or some combination thereof and can include any number of wired or wireless links. In general, communication over the network 480 can be carried via any type of wired and/or wireless connection, using a wide variety of communication protocols (e.g., TCP/IP, HTTP, SMTP, FTP), encodings or formats (e.g., HTML, XML), and/or protection schemes (e.g., VPN, secure HTTP, SSL).

FIG. 4 illustrates one example computing system that can be used to implement the present disclosure. Other computing systems can be used as well. For example, in some implementations, the footwear device 402 can include the model trainer 460 and the training dataset 462. In such implementations, the models 420 can be both trained and used locally at the footwear device 402. In some of such implementations, the footwear device 402 can implement the model trainer 460 to personalize the models 420 based on user-specific data.

### Example Methods

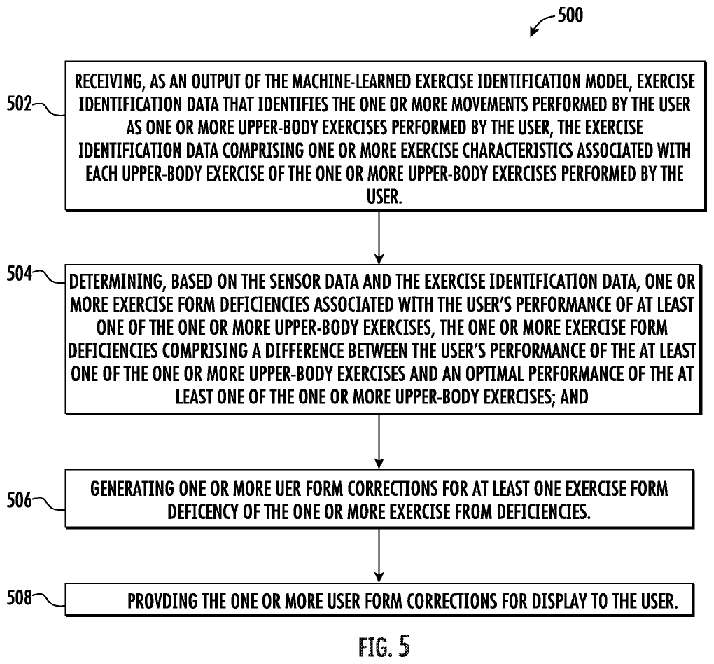

FIG. 5 is a flowchart depicting an example method of generating user form corrections for user form deficiencies in accordance with example embodiments of the present disclosure. The method 500 can be implemented, for instance, using computing device(s) of FIG. 1 (e.g., FIG. 1B-1D). FIG. 5 depicts steps performed in a particular order for purposes of illustration and discussion. Those of ordinary skill in the art, using the disclosures provided herein, will understand that various steps of any of the methods described herein can be omitted, rearranged, performed simultaneously, expanded, and/or modified in various ways without deviating from the scope of the present disclosure.

At 502, the method 500 can include receiving, as an output of the machine-learned exercise identification model, exercise identification data that identifies one or more movements performed by the user as one or more upper-body exercises performed by the user. The exercise identification data can include one or more exercise characteristics associated with each upper-body exercise of the one or more upper-body exercises performed by the user. Exercise characteristics can describe and/or indicate one or more characteristics (e.g., number of repetitions performed, amount of weight used, number of sets performed, amount of rest time used, etc.) associated with one or more upper-body exercises.

At 504, the method 500 can include determining, based on the sensor data and the exercise identification data, one or more exercise form deficiencies associated with the user's performance of at least one of the one or more upper-body exercises. The exercise form deficiencies can be determined based on the sensor data and the exercise identification data (e.g., weight distribution of the user, force applied by the user, user limb positioning, amount of time, etc.). The exercise form deficiencies can include a difference between the user's performance of the at least one of the one or more upper-body exercises and an optimal performance of the at least one of the one or more upper-body exercises.

As an example, an optimal form for a clean exercise can include a knee positioning that does not extend over the vertical plane of the user's toe. If the user extends his knee past the vertical plane of the toe during the performance of a clean exercise, an exercise form deficiency can be determined based on a difference between the optimal knee positioning and the users knee positioning. As another example, an optimal form for a bench press can include an optimal amount of force applied to the ground. If the user fails to apply this amount of force to the ground during the performance of a bench press exercise, an exercise form deficiency can be determined based on a difference between the optimal amount of force applied to the ground and the user's amount of force applied to the ground.

At 506, the method 500 can include generating one or more user form corrections for at least one exercise form deficiency of the one or more exercise form deficiencies. As an example, for an exercise form deficiency associated with the user extending their knee over the vertical plane of the toe while performing a clean exercise, a user form correction can be generated that advises the user to place more weight on the heel of their foot while performing a clean exercise. As another example, for an exercise form deficiency associated with the user not applying enough force to the ground while performing a bench press exercise, a user form correction can be generated that advises the user to apply more force to the ground with their feet while performing a bench press exercise.

At 508, the method 500 can include providing the one or more user form corrections for display to the user (e.g., through an application associated with the footwear device, through a user display device, through tactile feedback, through audio feedback, etc.). As an example, the footwear device 102 can provide the one or more user form corrections to the ancillary computing device 130 (e.g., a user computing device such as a smartphone, etc.).

FIG. 6 is a flowchart depicting an example method for implementing pre-processing and feature extraction of sensor data in accordance with example embodiments of the present disclosure. The method 600 can be implemented, for instance, using computing device(s) of FIG. 1 (e.g., FIG. 1B-1D). FIG. 6 depicts steps performed in a particular order for purposes of illustration and discussion. Those of ordinary skill in the art, using the disclosures provided herein, will understand that various steps of any of the methods described herein can be omitted, rearranged, performed simultaneously, expanded, and/or modified in various ways without deviating from the scope of the present disclosure.

At 602, the method 600 includes obtaining sensor data generated by one or more sensors of a footwear device worn by a user. The one or more sensors can be positioned in one or more positions of the footwear device. The sensor data can be associated with one or more movements performed by the user.

At 604, the method 600 includes applying a filter to the data channels of the one or more sensors of the footwear device. As an example, a filter (e.g., band-pass filter, high-pass filter, low-pass filter, etc.) can be applied to the data channel of each pressure sensor. As another example, a filter can be applied to a data channel of a gyroscope and accelerometer. In some implementations, the filter can be configured to remove drift signal and/or high frequency noise from the data channels. As an example, the filter can be configured to remove drift signal below 0.2 Hz and remove high-frequency noise above 3 Hz. It should be noted that any sort of filter can be applied to the data channel(s) of sensor(s) for attenuation and/or amplification. It should be noted that applying a filter to the data channels of the one or more sensors can increase the quality of sensor data, but is not necessarily required.

At 606, the method can include extracting one or more features from the data channels of the one or more sensors. The one or more features can include statistical feature(s) (e.g., mean, median, absolute median, variance, root mean square, entropy, etc.), frequency feature(s) (e.g., power spectrum density, dominant frequency, dominant frequency energy, DC offset, etc.), and/or auto-correlation feature(s) (e.g., maximum autocorrelation peak, number of autocorrelation peaks, height of a first autocorrelation peak after a first zero crossing, etc.). As an example, 14 different features can be extracted from each data channel of each sensor of a footwear device. It should be noted that feature extraction can be implemented additionally or alternatively to implementation of data filtering. Similarly to the implementation of data filtering, feature extraction from data channel(s) can increase machine-learned model performance but is not necessarily required.

### Additional Disclosure

The technology discussed herein makes reference to servers, databases, software applications, and other computer-based systems, as well as actions taken and information sent to and from such systems. The inherent flexibility of computer-based systems allows for a great variety of possible configurations, combinations, and divisions of tasks and functionality between and among components. For instance, processes discussed herein can be implemented using a single device or component or multiple devices or components working in combination. Databases and applications can be implemented on a single system or distributed across multiple systems. Distributed components can operate sequentially or in parallel.

While the present subject matter has been described in detail with respect to various specific example embodiments thereof, each example is provided by way of explanation, not limitation of the disclosure. Those skilled in the art, upon attaining an understanding of the foregoing, can readily produce alterations to, variations of, and equivalents to such embodiments. Accordingly, the subject disclosure does not preclude inclusion of such modifications, variations and/or additions to the present subject matter as would be readily apparent to one of ordinary skill in the art. For instance, features illustrated or described as part of one embodiment can be used with another embodiment to yield a still further embodiment. Thus, it is intended that the present disclosure cover such alterations, variations, and equivalents.

1. A computing system, comprising:
one or more processors; and
one or more non-transitory computer-readable media that store instructions that when executed by the one or more processors cause the computing system to perform operations, the operations comprising:
obtaining sensor data generated by one or more sensors of a footwear device worn by a user, the one or more sensors positioned in one or more positions of the footwear device, the sensor data associated with one or more movements performed by the user;
inputting the sensor data into a machine-learned exercise identification model; and
receiving, as an output of the machine-learned exercise identification model, exercise identification data that identifies the one or more movements performed by the user as one or more upper-body exercises performed by the user. the exercise identification data comprising one or more exercise characteristics associated with each upper-body exercise of the one or more upper-body exercises performed by the user.

2. The computing system of claim 1, wherein the one or more exercise characteristics comprises an amount of time spent performing each upper-body exercise of the one or more upper-body exercises performed by the user.

3. The computing system of claim 1. wherein the one or more exercise characteristics comprises an exercise categorization that further categorizes at least one exercise of the one or more upper-body exercises performed by the user as a full-body exercise.

4. The computing system of claim 1, wherein at least one of the one or more exercise characteristics comprises a number of sets performed for each upper-body exercise of the one or more upper-body exercises performed by the user.

5. The computing system of claim 4, wherein the one or more exercise characteristics comprises a number of repetitions performed for each set performed for each upper-body exercise of the one or more upper-body exercises performed by the user.

6. The computing system of claim 1. wherein the one or more exercise characteristics comprises a weight used for each upper-body exercise of the one or more upper-body exercises performed by the user.

7. The computing system of claim 1, wherein the operations further comprise:
determining, based on the sensor data and the exercise identification data, one or more exercise form deficiencies associated with the user's performance of at least one of the one or more upper-body exercises, the one or more exercise form deficiencies comprising a difference between the user's performance of the at least one of the one or more upper-body exercises and an optimal performance of the at least one of the one or more upper-body exercises; and
generating one or more user form corrections for at least one exercise form deficiency of the one or more exercise form deficiencies.

8. The computing system of claim 7, wherein the operations further comprise:
providing the one or more user form corrections for display to the user.

9. The computing system of claim 1, wherein the one or more sensors comprise at least one of:
a pressure sensor;
an accelerometer;
a gyroscope;
an inertial measurement unit; or
a force-sensitive resistor.

10. The computing system of claim 9, wherein the pressure sensor comprises a barometer and a rubber layer, the barometer positioned at least one of below or inside the rubber layer.

11. The computing system of claim 1, the operations further comprising:
evaluating a loss function that evaluates a difference between the exercise identification data and actual exercise data; and
modifying values for one or more parameters of the machine-learned exercise identification model based on the loss function.

12. The computing system of claim 11, wherein the difference between the exercise identification data and the actual exercise data comprises at least one of a difference in pressure or a difference in force: and
wherein the actual exercise data is provided by the user.

12. (canceled)

13. The computing system of claim 1, wherein the machine-learned exercise identification model includes a convolutional neural network.

14. A footwear device, comprising:
one or more sensors;
one or more processors; and
one or more non-transitory computer-readable media that store instructions that when executed by the one or more processors cause the footwear device to perform operations, the operations comprising:
obtaining sensor data generated by the one or more sensors, the one or more sensors positioned in one or more positions of the footwear device, the sensor data associated with one or more movements performed by a user wearing the footwear device;
inputting the sensor data into a machine-learned exercise identification model; and
receiving, as an output of the machine-learned exercise identification model, exercise identification data that identifies the one or more movements performed by the user as one or more upper-body exercises performed by the user, the exercise identification data comprising one or more exercise characteristics associated with each upper-body exercise of the one or more upper-body exercises performed by the user.

15. The footwear device of claim 14, wherein the one or more sensors comprise a pressure sensor that comprises a barometer and a rubber layer, the barometer positioned at least one of below or inside the rubber layer.

16. The footwear device of claim 15, wherein the sensor data comprises a combination of pressure data and force data.

17. The footwear device of claim 16, wherein the one or more sensors comprise at least four pressure sensors and at least one inertial measurement unit sensor.

18. The footwear device of claim 17, wherein the footwear device comprises a shoe, a sock, an insertable insole, or an external shoe covering.

19. The footwear device of claim 18, wherein four pressure sensors of the at least four pressure sensors are respectively placed at an anterior toe area of the footwear device, a posterior heel area of the footwear device, a left-side area of the footwear device and a right-side area of the footwear device.

20. A computer-implemented method to perform exercise identification, the method comprising:
obtaining, by one or more computing devices, sensor data generated by one or more sensors of a footwear device worn by a user, the one or more sensors positioned in one or more positions of the footwear device, the sensor data associated with one or more movements performed by the user;
inputting, by the one or more computing devices, the sensor data into a machine-learned exercise identification model; and
receiving, by the one or more computing devices and as an output of the machine-learned exercise identification model, exercise identification data that identifies the one or more movements performed by the user as one or more upper-body exercises performed by the user, the exercise identification data comprising one or more exercise characteristics associated with each upper-body exercise of the one or more upper-body exercises performed by the user.
